(19) United States
(12) Patent Application Publication
(10) Pub. No.: US 2009/0175758 A1
(43) Pub. Date: Jul. 9, 2009

(54) WASTEWATER TANK STORAGE TREATMENT

(75) Inventors: Mary R. BURROWS, Pinckney, MI (US); M. Karin JOHANSSON, Saline, MI (US)

Correspondence Address: HARNESS, DICKEY & PIERCE, P.L.C., P.O. BOX 828, BLOOMFIELD HILLS, MI, 48303, US

(73) Assignee: THETFORD CORPORATION, Ann Arbor, MI (US)

(21) Appl. No.: 12/331,976

(22) Filed: Dec. 10, 2008

Related U.S. Application Data
(60) Provisional application No. 61/019,068, filed on Jan. 4, 2008.

(51) Int. Cl. A61L 2/18; A61L 11/00

(52) U.S. Cl. 422/5; 424/76.5

(57) ABSTRACT

A method and composition for treating a wastewater storage tank during non-use. The method comprises adding a storage deodorant composition to a wastewater storage tank, the storage deodorant composition comprising an odor control agent, a detergent, a surface active agent, and a lubricant; and leaving the storage composition in the wastewater storage tank for a period of 1 day to 12 months.

### CROSS-REFERENCE TO RELATED APPLICATIONS

This application claims priority to U.S. Provisional Patent Application No. 61/019,068 filed 4 Jan. 2008, which application is herein expressly incorporated by reference.

### FIELD

The present disclosure relates to storage deodorant compositions and methods for using such compositions to deodorize and store wastewater storage tanks while not in use.

### BACKGROUND

The statements in this section merely provide background information related to the present disclosure and may not constitute prior art.

Recreational vehicles (RV), portable toilets, tote tanks, sewage truck tanks, airplane bathrooms and other temporary bathroom facilities often employ one or more wastewater storage tanks to store waste from sanitary fixtures, sinks, faucets and other wastewater sources.

In an RV plumbing system, for example, there are separate wastewater storage tanks for what is referred to in the RV industry as “gray water” and “black water”. Gray water is water that passes through sinks, and showers. Black water is water that passes through toilets.

Within the context of the self-contained RV, the gray water tanks and the black water tanks are typically located exterior to the living space of the RV. Typically, they store wastewater and periodically require emptying (e.g., every 3-7 days) during normal use. The tanks themselves are not normally removed unless they are taken for service. The water tanks generally have their own outlets on the exterior of the RV and are controlled by independent valves. When the wastewater storage tank requires emptying, the owner has the option of dispensing the contents of the wastewater storage tank to an intermediate container or taking the RV to a specifically designated waste disposal site.

At the end of an RV season or when it is otherwise anticipated that the portable toilet will not in use for an extended period, a shut-down and storage procedure is usually performed. Typically, the contents of the gray water and black water tanks are emptied into a disposal site and the tanks are flushed with fresh water. In colder climates, these tanks and the connected plumbing parts such as seals, outlets, tubing and gate and/or dump valves must be exposed to antifreeze solution to prevent cracking and damage.

Often after an antifreeze solution has been flushed, the tanks are left for storage. This may result in odors, waste residue adhered to the tank, and valves that are difficult to operate after sitting for a period of time.

The above problems are significant in the use of portable wastewater storage tanks and containers. There remains a need to provide a product designed for use during storage having a deodorizing effect, a cleaning effect and a lubrication effect.

### SUMMARY

The present disclosure provides a method for treating a wastewater storage tank during non-use, the method comprising: (a) adding a storage deodorant composition to a wastewater storage tank, the storage deodorant composition comprising an odor control agent, a detergent, a surface active agent, and a lubricant; and (b) leaving the storage composition in the wastewater storage tank for a period (i.e., 1 day to 12 months) before removing the storage composition from the wastewater storage tank.

Compositions are also provided for the deodorization and storage of wastewater storage tanks. The storage deodorant composition of the present disclosure functions to both deodorize the wastewater storage tank and provide lubrication to valves, seals and other plumbing components of the tanks. Furthermore, the compositions comprise (a) an odor control agent; (b) a detergent; (c) a surface active agent; and (d) a lubricant. In some embodiments, the composition can also optionally contain pH modifiers, viscosity control agents, dyes and perfumes. The composition of the present disclosure is compatible with antifreeze agents.

Further areas of applicability will become apparent from the description provided herein. It should be understood that the description and specific examples are intended for purposes of illustration only and are not intended to limit the scope of the present disclosure.

### DETAILED DESCRIPTION

The following description is merely exemplary in nature and is not intended to limit the present disclosure, application, or uses.

The present disclosure relates to storage compositions for deodorizing and storing a wastewater storage tank including a holding tank or a tote tank found in an RV and other vessels, such as boats and aircraft, portable toilets, and where tanks are utilized to store wastewater originating from toilets, sinks, showers faucets and the like. As used herein, a wastewater storage tank includes tanks made from various materials, including plastic, metal, ceramic and other materials commonly used in the manufacture of wastewater storage tanks. Wastewater storage tanks also include black water and gray water RV holding tanks, marine and boat holding tanks, aircraft lavatory holding tanks and holding tanks found in portable toilets.

In some embodiments, the storage deodorant composition comprises: (a) an odor control agent; (b) a detergent; (c) a surfactant and (d) a lubricant. Typically, the storage deodorant composition is compatible with antifreeze agents commonly used to treat and store the wastewater storage tanks and associated plumbing over the winter months where temperatures may fall near or below 0° C. (32° F.).

### Storage Deodorant Composition

The storage deodorant composition can be added to an empty wastewater storage tank or can be added while the tank is in use. In some embodiments, the composition can be formulated in liquid form, dry granular form, disintegrating tablet form, in powder form and combinations thereof. In some embodiments, the active ingredients of the wastewater storage composition when in a dry formulation can be formulated with various excipients in an immediate release or a modified release formulation, illustratively selected from delayed release, sustained release, timed release, and combinations thereof. In some embodiments of the present disclosure, reference will be made to a standard concentration composition. As used herein, the standard concentration composition can contain an amount of the dry components of the composition diluted to one gallon with a liquid, for example, water. The components of the composition are therefore referenced on a weight percentage of the standard concentration composition which can vary from 5.0 lb/gal. to about 10 lb/gal. In various embodiments, the standard concentration is calculated on a density of 8.478 lb/gal. Concentrates of the standard concentration composition can be made by either increasing the proportion of weight components in the same volume of water, and/or decreasing the volume of water to make up the concentrated solution. Amounts of each of the components are presented as a percent by weight of the standard concentration composition unless otherwise noted.

### Detergents

Suitable detergents can include nonionic, cationic, anionic, amphoteric, and zwitterionic surfactants. Examples of suitable nonionic surfactants include but are not limited to alcohol ethoxylates, alkylphenol ethoxylates, phenol ethoxylates, amide ethoxylates, glyceride ethoxylates (soy bean oil and caster oil ethoxylates), fatty acid ethoxylates, alkyl glycosides and fatty amine ethoxylates.

In some embodiments, alkoxylated alcohols having a hydrophilic lipophilic balance (HLB) of about 10-16 moles ratio with alcohol are suitable for the detergent component. In some embodiments, the alkoxylated alcohol can be a C₁₁7 to C₁₁15 polyethoxylated alcohol commercially available as Tomadol 1-9 C₁₁ with about 9 moles of ethylene oxide from Tomah Products Incorporated (Milton, Wis., USA) or Neodol 25-12 from Shell Chemicals (Houston, Tex., USA) (condensation product of C₁₂-C₁₅ linear alcohols with an average of about 12 moles of ethylene oxide). Other suitable ethoxylated alcohols include TERGITOL 15-S-9 (the condensation product of C₁₁-C₁₅ linear alcohols with an average of about 9 moles of ethylene oxide), marketed by Dow Chemical Corporation; and NEODOL 23-6.5T (condensation product of (C₁₂-C₁₃) linear alcohols with an average of about 6.5 moles of ethylene oxide that have been distilled (topped) to remove certain impurities, and the PLURAFAC brand name surfactants marketed by BASF Corp. of Mount Olive, N.J., such as PLURAFAC A-38 (a condensation product of a C₁₈ straight chain alcohol with an average of about 27 moles of ethylene oxide). Other examples of ethoxylated alcohol surfactants are supplied by Imperial Chemical Company (ICI) of Wilmington, Del. These include the class of BRIJ surfactants and mixtures thereof, such as BRIJ 76 (i.e., Steareth-10) and BRIJ 56 (i.e., Ceteth-10). Other suitable nonionic surfactants for use in the present disclosure can also include alkylglycosides; alkylglycoside ethers as described in U.S. Pat. No. 4,011,389, issued to Langdon et al. on Mar. 8, 1977; alkylpolyethoxylated esters such as PEGOSPERSE 1000MS, available from Lonza Inc. of Fair Lawn, N.J.; ethoxylated sorbitan mono-, di- and/or tri-esters of C₁₂-C₁₈ i.e. 12-18 carbon fatty acids having an average degree of ethoxylation of about 2 to about 20, preferably about 2 to about 10, such as TWEEN 60 (sorbitan esters of stearic acid having an average degree of ethoxylation of about 20), TWEEN 20 (sorbitan esters of lauric acid having an average degree of ethoxylation of about 20) and TWEEN 61 (sorbitan esters of stearic acid having an average degree of ethoxylation of about 4). In some embodiments, a suitable surfactant for use in the present disclosure includes AEROSOL TO, a dioctyl ester of sodium sulfosuccinic acid marketed by Cytec Industries Inc. of West Paterson, N.J.

In some embodiments, the detergent component of the storage deodorant composition can contain an anionic surfactant at a concentration from about 1% to about 5% on a weight percentage basis. Anionic surfactants are known and can include alkylbenzene sulfonate surfactants and alkylether sulfate surfactants. Suitable anionic surfactants include those described in U.S. Pat. No. 2,220,099 and U.S. Pat. No. 2,477,383. Such surfactants include the linear straight chain alkylbenzene sulfonates averaging about 9 to about 16 carbon atoms in the alkyl chain and generally abbreviated as “LAS” including, but not limited to, decylbenzensulfonic acid, decylbenzensulfonate, dodecylbenzensulfonic acid, dodecylbenzensulfonate, tetradecylbenzensulfonic acid, tetradecylbenzensulfonate, undecylbenzensulfonic acid, undecylbenzensulfonate, nonylbenznesulfonic acid, nonylbenznesulfonate, hexdecylbenzensulfonic acid, and hexadecylbenzensulfonate. Suitable surfactants include the surfactants sold under the tradenames Biosoft S-100 available from Stepan, Calsoft LAS-99 available from Pilot, Phodacal LA Acid available from Rhone-Poulenc. Suitable alkali metals include sodium, potassium, and lithium. Suitable alkylamines include any short chain alkylamine, i.e., alkylamines wherein the carbon chain of the alkyl groups have no more than 6 carbon atoms. Useful alkylamines include monoethanolamine (MEA), 2-aminoethanol, 1-aminopropanol, 2-amino-methyl-1-propanol (AMP-95), 2-aminopropanol, triethylamine, triethanolamine (TEA), diethanolamine (DEA), diethylamine, triphenylamine, and mixtures thereof. The sulfonic group of the benzenesulfonic acid-based anionic surfactants should be neutralized by about 50% using alkylamine. In particular, the sulfonic group can be neutralized by about 50% to about 100% using alkylamine. An anionic surfactant is considered 50% neutralized when alkyl amine is present at a 1:2 mole ratio of alkylamine:surfactant at a pH of about 7 or higher.

In some embodiments, the pH of the composition is below 7.0 and can be maintained at an acidic pH with benzenesulfonic acid-based anionic surfactants.

In some embodiments, the alkylether sulfate surfactants can include any alcohol alkoxysulfate anionic surfactant. In some embodiments, R₂ can vary within any one molecule of the alcohol alkoxysulfate surfactant such that the molecule can contain ethoxy, propoxy, butoxy groups, or a mixture thereof. Methods for manufacturing alkylether sulfates are known. Briefly, alkylether sulfate surfactants can be manufactured by condensing an alcohol with ethylene oxide followed by sulfonation and neutralization. Suitable alkylether sulfate surfactants can include the surfactants sold under the tradenames Avirol and Stanpol 230-E available from Henkel, Geropan available form Rhone-Polanc, Calform available from Pilot, and Polystep and Steol available from Stepan.

Other anionic surfactants include alkyl glyceryl ether sulfates or alkyl glyceryl ether sulfonates, which are typically manufactured by condensing an alcohol with ethylene oxide followed by sulfation process and neutralization. Suitable surfactants include PEG (1-4) dodecylsulfate, (ammonium salt) PEG (12) decylsulfate (ammonium salt), PEG (9) dodecylsulfate (ammonium salt), PEG (12) dodecylsulfate, sodium salt, PEG (9) dodecylsulfate, sodium salt, PEG (1-4) dodecylsulfate, sodium salt, PEG (12) tetradecylsulfate, sodium salt, PEG (9) tetradecylsulfate, sodium salt, PEG (1-4) dodecylsulfate, sodium salt, PEG (12) dodecylsulfate, potassium salt, PEG (12) dodecylsulfate, magnesium salt, PEG (9) dodecylsulfate and sodium salt.

In some embodiments, the detergent component of the storage deodorant composition can be present in an amount of about 10% or less of the standard concentration composition based on a density of 8.478 lb/gal. In some embodiments the detergent component of the storage deodorant composition can be present in an amount of about 0.01% to about 10% by wt. In some embodiments, the amount of the detergent in the storage deodorant composition is at least about 0.01%, at least about 0.1%, at least about 0.2%, at least about 0.5%, at least about 1.0% or at least about 1.5%, and less than about 8%, less than about 6% less than about 5% less than about 3%, less than about 2.5% by weight of the standard concentration composition. In some embodiments, the amount of detergent present in the composition can be about 0.1% to about 10% by weight, 1 to 5% by weight and from about 2-3% by weight of the standard concentration composition having a final density of 8.478 LB/gal. (1.02 kg/L).

### Odor Control Agents

In some embodiments of the present disclosure, the deodorant storage composition also comprises one or more odor control agents. In some embodiments, the odor control agents can include any chemical agent known to eliminate the source of malodor, absorb the malodor, and mask the malodor and combinations thereof. In some embodiments, the odor control agent can include one or more biocides. Biocides of the present disclosure can include any bactericide, viricide, fungicide, algaecide, pesticide, herbicide and combinations thereof. The present disclosure also contemplates the use of anti-biofouling agents that are known in the fields of medicine, veterinary medicine, agriculture, commercial transportation, textile, paper manufacture, water treatment, food preparation services and personal care chemistry.

In some embodiments, the odor control agent can include a biocide, for example oxine and derivatives thereof; chlorophenols such as tri-, tetra- and pentachlorophenol; anilides such as 2,5-dimethyl-3-furylanilide; benzimidazoles such as 2-(methoxy-carbamoyl)benzimidazole; nitrophenols such as dinitrophenol; nitrocresols such as dinitrocresol; crotonates such as 2,4-dinitro-6-(2-octyl)phenyl crotonate; organotins such as triphenyltin acetate and tributyl tinoxide; antibiotics such as streptomycin and griseofulvin; organic acids such as acetic, sorbic, salicylic, benzoic, dehydroacetic and undecylenic acids; oxathiius such as 5,6-dihydro-2-methyl-1,4-oxathiin-3-carboxanilide; sulfones such as diiodomethyl-p-tolyl sulfone; iodine, compounds thereof, and iodofors; aliphatic alcohols from C₁ to C₁₈ chain length; alkyl, benzyl and phenyl alcohols for example cresols; hydroxyl diphenyl oxide and sulfide isomers; carbanilides such as trichlorocarbanilide; bis phenols such as hexachlorophene; phenyl mercurials such as phenyl mercuric acetate and nitrate; complex organic mercurials such as thimersol and nitromersol and aldehydes such as formaldehyde, paraformaldehyde, oxaldehyde and glutaraldehyde.

In some embodiments, biocides of the present disclosure can include any bactericide, viricide, fungicide, algaecide, pesticide, herbicide and combinations thereof. The present disclosure also contemplates the use of anti-biofouling agents that are known in the fields of medicine, veterinary medicine, agriculture, commercial transportation, textile, paper manufacture, water treatment, food preparation services and personal care chemistry.

Illustrative examples of biocides can include narrow and/or broad spectrum bactericides that are used to control, reduce, or inhibit the growth of microorganisms. In some embodiments, the biocide can include one or more of 2-Bromo-2-nitro-1,3-propanediol sold under the trade name Protectol® BN; 2-Bromo-2-nitro-1,3-propanediol sold under the trade name Protectol® BN 30; 2-Bromo-2-nitro-1,3-propanediol sold under the trade name Protectol® BN 18; 1,5 Pentanedial (Glutaraldehyde) sold under the trade name Protectol® GA 50; 1,5 Pentanedial (Glutaraldehyde) sold under the trade name Protectol® GA 24; 2-Phenoxyethanol sold under the trade name Protectol® PE/PE S; 1-Phenoxy-2-propanol sold under the trade name Protectol® PP; 1,3,5-Triazine-1,3,5(2H, 4H,6H)-triethanol (Triazine) sold under the trade name Protectol® HT; Tetrahydro-1,3,4,6-tetrakis(hydroxymethyl)-imidazol[4,5-d]-imidazole-2,5(1H,3H)-dione sold under the trade name Protectol® TD; 1,2-Ethanedial (Oxaldehyde or Glyoxal) sold under the trade name Protectol® GL; Tetrahydro-2,5-dimethoxyfuran sold under the trade name Protectol® DF; 2,4-Dichlorobenzenemethanol sold under the trade name Protectol® DA/DA S; Tetrahydro-3,5-dimethyl-2H-1,3,5-thiadiazine-2-thione sold under the trade name Protectol®DZ; and Tetrahydro-3,5-dimethyl-2H-1,3,5-thiadiazine-2-thione.

In some embodiments, the biocide can include a quaternary ammonium agent, for example quaternary ammonium compounds. Non-limiting examples of these quaternary compounds can include benzalkonium chlorides and/or substituted benzalkonium chlorides, di(C₆-C₁₄)alkyl di short chain (C₁₋₄ alkyl and/or hydroxyalki) quaternary ammonium salts, N-(3-chloroallyl)hexaminium chlorides, benzethonium chloride, methylbenzethonium chloride, and cetylpyridinium chloride. Other quaternary compounds can include dialkyldimethyl ammonium chlorides, alkyl dimethylbenzyl ammonium chlorides, dialkylmethylbenzyl ammonium chlorides, octyl decyidimethyl ammonium chlorides, dioctyl dimethyl ammonium chlorides, didecyl dimethyl ammonium chlorides and mixtures thereof. Biguanide biocides including, but not limited to polyhexamethylene biguanide hydrochloride, p-chlorophenyl biguanide; 4-chlorobenzhydryl biguanide, halogenated hexidine such as, but not limited to, chlorhexidine(1,1′-hexamethylene-bis-5-(4-chlorophenyl biguanide) and its salts are also contemplated. The quaternary ammonium compounds are commercially available from companies including TCI, Portland Oreg., USA; Stepan, Northfield, Ill., USA and Mason Chemical Company, Arlington Heights, Ill., USA.

In some embodiments, the biocide can include a formaldehyde containing agent, for example formaldehyde, oxaldehyde(ethanedial)paraformaldehyde and formaldehyde donor compounds such as 5,5-disubstituted hydantoins include 5,5-dimethylhydantoin (DMH), 5-methyl-5-ethylhydantoin, 5,5diethylhydantoin, 5,5-diphenylhydantoin, 5-methyl-5phenylhydantoin, 5,5-pentamethylenehydantoin, 1,3-dimethylol-5,5-dimethylhydantoin and the like known in the art as possessing broad spectrum bactericidal or fungicidal activity.

In some embodiments, other useful odor control agents can also include calcium containing agents, for example, calcium nitrate salts. Other classes of useful odor control agents can include oxidizers commonly used in disinfecting agents and are compatible with antifreeze solutions.

In some embodiments, the odor control agent of the storage deodorant composition can be present in an amount of about 10% or less of the standard concentration composition based on a weight of 8.478 lb/Gal. In some embodiments the odor control agent component of the storage deodorant composition can be present in an amount of about 0.01% to about 10% by wt. In some embodiments, the amount of the detergent in the storage deodorant composition is at least about 0.01%, at least about 0.1%, at least about 0.2%, at least about 0.5%, at least about 1.0%, and less than about 8%, less than about 6% less than about 5% less than about 3%, less than about 2% by weight of the standard concentration composition. In some embodiments, the amount of detergent present in the composition can be about 0.1% to about 10% by weight, 1 to 5% by weight and from about 2-3% by weight of the standard concentration composition having a final weight of 8.478 LB/Gal. (1.02 kg/L).

### Surface Active Agent

In some embodiments, the storage composition further contains at least one surface active agent. In some embodiments, the surface active agent can be one or more alkyl sulfates. Preferred salts are the Na, K, TEA (Triethanolamine) and NH₄⁺ salts. Mixtures of these salts can be used. In some embodiments, the alkyl sulfates include sodium dodecyl sulfate (also known as sodium lauryl sulfate) ammonium alkyl sulfates and the like; alkyl ether sulfates, for example sodium laureth sulfate and fatty acid ester sulfates, for example cocoyl glyceride sulfate and the like. The alkyl sulfates are commercially available from Lankem Ltd., Cheshire, UK.

In some embodiments, the surface active agent can also include one or more ethoxylated alkyl alcohols, for example, ethoxylated tridecyl alcohol. Other agents can include, EO/PO block copolymers, and sulfonated alkyl esters, for example, sulfonated methyl esters.

In some embodiments, the surface active agent of the storage deodorant composition can be present in an amount of about 5% or less of the standard concentration composition based on a density of 8.478 lb/gal. In some embodiments, the surface active agent of the storage deodorant composition can be present in an amount of about 0.01% to about 5% by wt. In some embodiments, the amount of the surface active agent in the storage deodorant composition is at least about 0.01%, at least about 0.1%, at least about 0.2%, at least about 0.5%, at least about 1.0%, at least about 1.5% and less than about 5%, less than about 4%, less than about 3%, less than about 2% by weight of the standard concentration composition. In some embodiments, the amount of surface active agent present in the composition can be about 0.01% to about 5% by weight, from about 0.5% to about 4% by weight and from about 0.75% to about 3% by weight of the standard concentration composition having a final density of 8.478 LB/Gal. (1.02 kg/l).

### Lubricant

The deodorant storage composition of the present disclosure also provides for enhanced lubrication of the tank and dump valves, pour spouts, seals, pipes and associated plumbing parts. In some embodiments, the storage deodorant composition also includes a lubricant. The lubricant in the deodorant storage composition can include any lubricant that is compatible with the antifreeze solutions commonly associated with potable water and wastewater systems designed for RV and marine use. These antifreeze solutions typically contain between 15% and 35% by weight of solution of propylene glycol (Cas#57-55-6).

In some embodiments, the lubricant can include polymers or mixtures of polymers including, polysiloxanes including polydimethylsiloxane; alkylene oxides, including polyoxyalkylene ether, and combinations thereof. Other useful lubricants can also include phosphate esters and silicone glycols.

Still other types of suitable lubricants for use in the present disclosure can include silicone copolymers such as those made by Momentive Performance Materials of Fairfield, Conn. Illustrative silicone copolymers can include Momentive Performance Materials SF1188A (a copolymer of a polydimethylsiloxane and a polyoxyalkylene ether) and General Electric's SF1228 (a silicone polyether copolymer).

In some embodiments, the lubricant of the storage deodorant composition can be present in an amount of about 5% or less of the standard concentration composition based on a density of 8.478 lb/gal. In some embodiments, the lubricant component of the storage deodorant composition can be present in an amount of about 0.01% to about 5% by wt. In some embodiments, the amount of the lubricant in the storage deodorant composition is at least about 0.01%, at least about 0.1%, at least about 0.2%, at least about 0.5%, at least about 1.0%, at least about 1.5%, and less than about 5%, less than about 4%, less than about 3%, less than about 2% by weight of the standard concentration composition. In some embodiments, the amount of lubricant present in the composition can be about 0.01% to about 5% by weight, from about 0.1% to about 4% by weight and from about 0.5% to about 3% by weight of the standard concentration composition having a final density of 8.478 LB/gal. (1.02 kg/l).

### pH Modifiers

In some embodiments, the composition of the present disclosure may also include one or more pH modifiers to buffer the composition in the acidic or basic pH range. In some embodiments, the requirement for an acid or basic composition pH may depend on several factors, including the pH activity preference of the active agents, the compatibility between the pH of the storage deodorant composition and the antifreeze treatment, and the resistivity of the wastewater storage tanks to prolonged exposure to an acid or base pH.

In some embodiments, the pH modifier can be a weak or strong acid, for example, citric acid, acetic acid, ascorbic acid, boric acid, butanoic acid, carbonic acid, formic acid, heptanoic acid, hexanoic acid, hydrobromic acid, hydrochloric acid, hydrocyanic acid, hydrofluoric acid, hydroiodic acid, lactic acid, nitric acid, nitrous acid, octanoic acid, oxalic acid, perchloric acid, pentanoic acid, phosphoric acid, sulfuric acid, and sulfurous acid.

In some embodiments, the pH modifier can be a strong or weak base or basic agent. In some embodiments, the strong base can be any metal hydroxide, for example potassium, sodium, lithium, cesium, strontium, calcium, barium hydroxides. In some embodiments, weak base pH modifiers can include ammonia, alanine, dimethylamine, ethylamine, glycine, hydrazine, methylamine and trimethylamine.

In some embodiments, the pH modifier can also include a buffering agent compatible with an added acid. In one preferred embodiment, the pH modifier can be an acid, for example, citric acid and a second pH modifier can include sodium citrate dihydrate. Other compatible acid/base combinations can include acetic acid/sodium acetate, carbonic acid/potassium bicarbonate, phosphoric acid/potassium phosphate dihydrate, other acid/conjugate base combinations are well known in the chemical formulation arts. In some embodiments, it is generally preferred to keep the pH of the storage deodorant composition below 7.0 if propanediol is used as the odor control agent. For various embodiments, the storage deodorant solution has a basic pH that enables better detergency activity of its detergents and surface active agents.

In some embodiments, the one or more pH modifying agents of the storage deodorant composition can be present in an amount of about 5% or less of the standard concentration composition based on a weight of 8.478 lb/gal. In some embodiments, the one or more pH modifying agent of the storage deodorant composition can be present in an amount of about 0.01% to about 5% by wt. In some embodiments, the amount of the pH modifying agent in the storage deodorant composition is at least about 0.01%, at least about 0.1%, at least about 0.2%, at least about 0.5%, at least about 1.0%, at least about 1.5%, and less than about 5%, less than about 4%, less than about 3%, less than about 2% by weight of the standard concentration composition. In some embodiments, the amount of one or more pH modifying agent present in the composition can be about 0.01% to about 5% by weight, from about 0.25% to about 4% by weight and from about 0.5% to about 3% by weight of the standard concentration composition having a final density of 8.478 lb/gal. (1.02 kg/l).

### Perfumes

In some embodiments, the storage deodorant composition may also include one or more perfumes that are compatible with the above recited components, does not substantially compromise odor control activity and is compatible with antifreeze. The perfumes may be selected from the group consisting of ambrox, bacdanol, benzyl salicylate, butyl anthranilate, cetalox, damascenone, alpha-damascone, gamma-dodecalactone, ebanol, herbavert, cis-3-hexenyl salicylate, alpha-ionone, beta-ionone, alpha-isomethylionone, lilial, methyl nonyl ketone, gamma-undecalactone, undecylenic aldehyde, and mixtures thereof. The present invention may also include a perfume selected from the group consisting of allyl amyl glycolate, allyl caproate, amyl acetate, amyl propionate, anisic aldehyde, anisyl acetate, anisole, benzaldehyde, benzyl acetate, benzyl acetone, benzyl alcohol, benzyl formate, benzyl iso valerate, benzyl propionate, beta gamma hexenol, calone, camphor gum, laevo-carveol, d-carvone, laevo-carvone, cinnamic alcohol, cinnamyl acetate, cinnamic alcohol, cinnamyl formate, cinnamyl propionate, cis-jasmone, cis-3-hexenyl acetate, coumarin, cuminic alcohol, cuminic aldehyde, Cyclal C, cyclogalbanate, dihydroeuginol, dihydro isojasmonate, dimethyl benzyl carbinol, dimethyl benzyl carbinyl acetate, ethyl acetate, ethyl aceto acetate, ethyl amyl ketone, ethyl anthranilate, ethyl benzoate, ethyl butyrate, ethyl cinnamate, ethyl hexyl ketone, ethyl maltol, ethyl-2-methyl butyrate, ethyl methylphenyl glycidate, ethyl phenyl acetate, ethyl salicylate, ethyl vanillin, eucalyptol, eugenol, eugenyl acetate, eugenyl formate, eugenyl methyl ether, fenchyl alcohol, flat acetate(tricyclo decenyl acetate), fructone, frutene(tricyclo decenyl propionate), geraniol, geranyl oxyacetaldehyde, heliotropin, hexenol, hexenyl acetate, hexyl acetate, hexyl formate, hinokitiol, hydratropic alcohol, hydroxycitronellal, hydroxycitronellal diethyl acetal, hydroxycitronellol, indole, isoamyl alcohol, iso cyclo citral, isoeugenol, isoeugenyl acetate, isomenthone, isopulegyl acetate, isoquinoline, keone, ligustral, linalool, linalool oxide, linalyl formate, lyral, menthone, methyl acetophenone, methyl amyl ketone, methyl anthranilate, methyl benzoate, methyl benzyl acetate, methyl cinnamate, methyl dihydrojasmonate, methyl eugenol, methyl heptenone, methyl heptine carbonate, methyl heptyl ketone, methyl hexyl ketone, methyl isobutenyl tetrahydropyran, methyl-N-methyl anthranilate, methyl beta naphthyl ketone, methyl phenyl carbinyl acetate, methyl salicylate, nerol, nonalactone, octalactone, octyl alcohol (octanol-2), para-anisic aldehyde, para-cresol, para-cresyl methyl ether, para hydroxy phenyl butanone, para-methoxy acetophenone, para-methyl acetophenone, phenoxy ethanol, phenoxyethyl propionate, phenyl acetaldehyde, phenylacetaldehyde diethyl ether, phenylethyl oxyacetaldehyde, phenyl ethyl acetate, phenyl ethyl alcohol, phenyl ethyl dimethyl carbinol, prenyl acetate, propyl butyrate, pulegone, rose oxide, safrole, terpineol, vanillin, viridine, allyl heptoate, amyl benzoate, anethole, benzophenone, carvacrol, citral, citronellol, citronellyl nitrile, cyclohexyl ethyl acetate, cymal, 4-decenal, dihydro isojasmonate, dihydro myrcenol, ethyl methyl phenyl glycidate, fenchyl acetate, florhydral, gamma-nonalactone, geranyl formate, geranyl nitrile, hexenyl isobutyrate, alpha-ionone, isobornyl acetate, isobutyl benzoate, isononyl alcohol, isomenthol, para-isopropyl phenylacetaldehyde, isopulegol, linalyl acetate, 2-methoxy naphthalene, menthyl acetate, methyl chavicol, musk ketone, beta naphthol methyl ether, neral, nonyl aldehyde, phenyl heptanol, phenyl hexanol, terpinyl acetate, veratrol, yara-yara, and mixtures thereof.

In some embodiments, the perfume component of the storage deodorant composition can be present in an amount of about 5% or less of the standard concentration composition based on a density of 8.478 lb/gal. In some embodiments, the perfume component of the storage deodorant composition can be present in an amount of about 0.01% to about 5% by wt. In some embodiments, the amount of the perfume component in the storage deodorant composition is at least about 0.01%, at least about 0.1%, at least about 0.2%, at least about 0.5%, at least about 1.0%, at least about 1.5% and less than about 5%, less than about 4%, less than about 3%, less than about 1% by weight of the standard concentration composition. In some embodiments, the amount of surface active agent present in the composition can be about 0.01% to about 5% by weight, from about 0.1% to about 4% by weight and from about 0.3% to about 3% by weight of the standard concentration composition having a final density of 8.478 lb/gal. (1.02 kg/l).

In some embodiments, the storage deodorant compositions may also include one or more optional agents. In some embodiments, the storage deodorant composition may include a dispersion agent, for example, a hydrocolloid, a thickener, an emulsifier, a stabilizer and mixtures thereof. In some embodiments, the thickening agent can consist of a natural thickener for example, xanthan gum, acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, and mixtures thereof.

In some embodiments, the thickeners or gelling agents that may be included in the storage deodorant composition can comprise carboxylic acid polymers, wherein the polymers are crosslinked compounds containing one or more monomers derived from acrylic acid, substituted acrylic acids, and salts and esters of these acrylic acids and the substituted acrylic acids, and wherein the crosslinking agent contains two or more carbon-carbon double bonds and is derived from a polyhydric alcohol. In some embodiments illustrative examples of carboxylic acid polymers can include: carbomers, i.e. homopolymers of acrylic acid crosslinked with allyl ethers of sucrose or pentaerytritol. The carbomers are available as the Carbopol® 900 series from B.F. Goodrich (e.g., Carbopol® 954). In addition, other suitable carboxylic acid polymeric agents can include copolymers of C₁₀₋₃₀ alkyl acrylates with one or more monomers of acrylic acid, methacrylic acid, or one of their short chain (i.e., C₁₄ alcohol) esters, wherein the crosslinking agent is an allyl ether of sucrose or pentaerytritol. These copolymers are known as acrylates/C₁₀₋₃₀ alkyl acrylate crosspolymers and are commercially available as Carbopol®1342, Carbopol®1382, PEMULEN TR-1, and PEMULEN TR-2, from B.F. Goodrich. In other words, examples of carboxylic acid polymer thickeners useful herein are those selected from carbomers, acrylates/C.sub.10-C.sub.30 alkyl acrylate crosspolymers, and mixtures thereof.

In some embodiments, the optional thickeners or gelling agents may also include one or more crosslinked polyacrylate polymers. The storage deodorant compositions of the present disclosure can optionally contain crosslinked polyacrylate polymers including both cationic and nonionic polymers.

In some embodiments, the optional thickeners may also include one or more polyacrylamide polymers. The storage deodorant compositions of the present disclosure can optionally contain polyacrylamide polymers, especially nonionic polyacrylamide polymers including substituted branched or unbranched polymers. Other polyacrylamide polymers useful herein can include multi-block copolymers of acrylamides and substituted acrylamides with acrylic acids and substituted acrylic acids. Commercially available examples of these multi-block copolymers include HYPAN SR150H, SS500V, SS500W, SSSA100H, from Lipo Chemicals, Inc., (Patterson, N.J.).

In some embodiments, the thickening or gelling agent can also include a wide variety of polysaccharides other than the natural gums described above. “Polysaccharides” refer to gelling agents that contain a backbone of repeating sugar (i.e., carbohydrate) units. Non-limiting examples of polysaccharide gelling agents include those selected from cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl substituted celluloses. In these polymers, the hydroxy groups of the cellulose polymer is hydroxyalkylated (preferably hydroxyethylated or hydroxypropylated) to form a hydroxyalkylated cellulose which is then further modified with a C₁₀-C₃₀ straight chain or branched chain alkyl group through an ether linkage. Typically these polymers are ethers of C₁₀-C₃₀ straight or branched chain alcohols with hydroxyalkylcelluloses. Examples of alkyl groups useful herein include those selected from stearyl, isostearyl, lauryl, myristyl, cetyl, isocetyl, cocoyl (i.e. alkyl groups derived from the alcohols of coconut oil), palmityl, oleyl, linoleyl, linoleyl, ricinolyl, behenyl, and mixtures thereof. Preferred among the alkyl hydroxyalkyl cellulose ethers is the material given the CTFA designation cetyl hydroxyethylcellulose, which is the ether of cetyl alcohol and hydroxyethylcellulose. This material is sold under the tradename Natrosol® CS Plus from Aqualon Corporation (Wilmington, Del., USA).

Other useful polysaccharides include scleroglucans which are a linear chain of (1-3) linked glucose units with a (1-6) linked glucose every three units, a commercially available example of which is Clearogel™ CS11 from Michel Mercier Products Inc. (Mountainside, N.J., USA).

In some embodiments, the thickening agent, for example, a natural thickener of the storage deodorant composition can be present in an amount of about 5% or less of the standard concentration composition based on a density of 8.478 lb/Gal. In some embodiments, the thickening agent of the storage deodorant composition can be present in an amount of about 0.01% to about 2% by wt. In some embodiments, the amount of the thickening agent in the storage deodorant composition is at least about 0.01%, at least about 0.1%, at least about 0.2%, at least about 0.5%, at least about 1.0%, at least about 1.5% and less than about 5%, less than about 2%, less than about 1%, less than about 0.3% by weight of the standard concentration composition. In some embodiments, the amount of the thickening agent present in the composition can be about 0.01% to about 5% by weight, from about 0.1% to about 4% by weight and from about 0.3% to about 3% by weight of the standard concentration composition having a final density of 8.478 LB/Gal. (1.02 kg/L).

In some embodiments, the storage deodorant composition of the present disclosure may also include a dye and/or a colorant. In some embodiments, the dye can be a water soluble dye. The dye can be added to the composition in either a dry powder form or in a liquid form. Illustrative examples of water soluble dyes useful in the present invention include: Chromatint Violet D34020, Chromatint® Blue AZ 50%, Chromatint® Blue 0408, Chromatint® Blue 1454, Chromatint® Blue 1873 Chromatint® Violet 0930, Chromatint® Red 1064, Chromatint® Green 0454 Chromatint® Green 1121, Chromatint® Green 0262, Chromatint® Orange 0092, Chromatint® Yellow 1468 all of which are commercially available through Chromatech Inc. (Canton, Mich., USA).

In some embodiments, the dye of the storage deodorant composition can be present in an amount of about 1% or less of the standard concentration composition based on a density of 8.478 lb/Gal. In some embodiments, the dye of the storage deodorant composition can be present in an amount of about 0.0001% to about 1% by wt. In some embodiments, the amount of the dye in the storage deodorant composition is at least about 0.0001%, at least about 0.001%, at least about 0.005%, at least about 0.05%, less than about 1%, less than about 0.1%, less than about 0.02%, less than about 0.005% by weight of the standard concentration composition. In some embodiments, the amount of the dye present in the composition can be about 0.0001% to about 0.5% by weight, from about 0.0005% to about 0.01% by weight and from about 0.001% to about 0.009% by weight of the standard concentration composition having a final density of 8.478 LB/Gal. (1.02 kg/L).

### Methods of Use

In accordance with the present disclosure, methods are disclosed for use in reducing the malodor, providing cleaning and lubrication of various components in wastewater storage tanks during a period of storage. The present disclosure provides for a method of storing a wastewater storage tank or tote for a period of days to months, imparting benefits such as deodorization, cleaning and lubrication of valves, seals and other plumbing components. The present method also provides for a method for preventing malodor formation and damage to valves, seals and other plumbing components that is compatible with antifreeze treatments of the aforementioned wastewater storage tanks and totes.

In some embodiments, the present method can be applied to a variety of wastewater storage tanks, including those commonly found in RVs, boats, portable toilets, aircraft lavatory holding tanks and other tanks used for wastewater containment. In some embodiments, the method can be performed by adding a storage deodorant composition to a wastewater storage tank, the storage deodorant composition comprising an odor control agent, a detergent, a surfactant, and a lubricant; and (b) leaving the storage composition in the wastewater storage tank for a period of 1 day to 12 months before removing the storage deodorant composition from the wastewater storage tank.

In some embodiments, the composition can be added to the wastewater storage tank in a concentrated form to be diluted with other fluids in the wastewater storage tank, for example, antifreeze solution, or added to an empty tank and then diluting the composition with water. The storage deodorant composition of the present invention provides a deodorizing, cleaning and lubrication functionality that is directly compatible with antifreeze solutions designed for RV, marine, portable lavatories, aircraft bathroom uses. In some embodiments, a granular concentrated form of the composition can be added to water in the wastewater storage tank or alternatively, added to an equivalent volume of antifreeze solution in the tank to obtain an effective treatment dose.

In some embodiments, the storage deodorant composition can be formulated to provide effective deodorizing and lubricating activity for a period ranging from 1 day to 12 months before the storage deodorant composition in the unused tank requires replacement. In some embodiments, the activity period can last for at least 1 day, at least 1 week, at least 4 weeks, at least 6 weeks, at least 8 weeks, at least 10 weeks, at least 12 weeks, at least 4 months, at least 6 months, at least 8 months and at least 12 months.

In some embodiments, the operator of the black and gray wastewater storage tank or tote can prepare the tank for storage after a period of continuous use. In the case of an RV storage procedure, the operator can connect a sewer hose to the termination outlet. The sewer hose can be locked by locking the end levers over the termination end. The other end of the sewer hose can be connected to an approved dumping station outlet. The black tank termination valve can be opened to allow the contents of the black water tank to exit into the dumping station. Then the gray water tank termination valve can be opened to allow the contents of the gray water tank to be emptied. The termination valves can then be placed in a closed position and the sewer hose can be disconnected. Next, the black water and gray water termination valves can be opened and connected to a clean water hose and flushed with clean water. Next, the tanks can be emptied using the steps outlined above. A solution of antifreeze is added to the fresh water tank and pumped throughout the plumbing system and into the holding tank. Next, an effective amount of the storage deodorant solution can be poured directly into the wastewater tanks. In the case of tote tanks, the storage deodorant solution can be introduced into the tote tanks directly by pouring the storage deodorant solution through the sewer hose opening, alternatively, for RV's the storage deodorant solution can be poured into the toilet, sinks, tubs and basins directly to deliver the storage deodorant solution into the gray and black tanks.

In some embodiments, the effective amount of a standard storage deodorant concentration can depend on the size of the holding tank, tote or portable wastewater storage tank and the desired period of deodorization and storage required. In some embodiments, for a wastewater storage tank having an inner volume of 40 gallons and a storage period of 1-2 months requires a volume of standard storage deodorant concentration ranging between 150 to 300 mL, a period of 2-4 months requires a volume of standard storage deodorant concentration ranging between 400 to 550 mL a storage period of 5-6 months requires a volume of standard storage deodorant concentration ranging between 600 to 800 mL and a storage period of 8-12 months requires a volume of standard storage deodorant concentration of 1,000 to 1,200 mL.

The present storage deodorant composition can be formulated to a standard concentration having a specific gravity at 22° C. of 1.019 based on a density of 8.478 lb/gallon (1.02 kg/l) comprising about less than about 5% by wt % of an odor control agent, less than about 2% by wt % of a surface active agent, about 1% to about 5% of a detergent, about 0.1% to about 3% by wt % of citric acid, about 0.1% to about 2% by wt % of sodium citrate dihydrate, about 0.05% to about 2% by wt % of a lubricant, about 0.1% to about 1.5% of a perfume and about 79.5% to about 99% water. The composition optionally contains about 0.001% to about 0.5% by wt % of a viscosity control agent and about 0.0001% to about 0.1% of a dye or colorant.

A person of ordinary skill in the art will immediately recognize that concentrated formulations of the above standard composition can be formulated in ratios that are substantially similar to the ratio of components disclosed above for a standard concentration, by simply reducing the percentage of water used to prepare the concentrated form of the composition.

### EXAMPLES

### Example 1

In one embodiment, the storage deodorant composition of the present disclosure was formulated with the components recited in Table 1 below. Essentially, water was pumped into a mixing tank and mixing was commenced. An odor control agent and a surface active agent was then added to the water in the mixing tank and mixed for about 15 minutes. Detergent was then added into a slurry tank. A viscosity control agent and a perfume were then added to the slurry tank and mixed for about 5 minutes. The contents of the slurry tank were then added to the contents of the mixing tank and mixed in the mixing tank for about 1 hour. Citric acid and sodium citrate dihydrate were then added to the mixing tank and mixed for about 20 minutes. Finally, a lubricant and a violet dye were added to the mixing tank and mixed for a further 15 minutes.

TABLE 1
Recitation of the components and amounts used to make an embodiment of
the present storage deodorant composition in accordance to the present disclosure.

| Component | Wt % | LB/225 Gal. | Source | Trade name |
|---|---|---|---|---|
| water | 92.38 | 211.8 Gal. | City Water | |
| odor control agent | 1.5 | 28.6 | Dow, ISI, | Bioban BP Plus, Bronopol, Myacide |
| | | | Morre-TEC, | AS, Protectol BN98, Protectol 6N99 |
| | | | BASF, Bayer | and Preventol P-100 |
| surface active agent | 0.9 | 17.2 | Cognis | Texapon V95G |
| detergent | 2.0 | 38.2 | Tomah Prod., | Tomadol 1-9 95%, Bio-Soft N1-9 |
| | | | Stepan Co. | 95% |
| lubricant | 0.75 | 14.3 | Momentive | SF1188A |
| | | | Performance | |
| | | | Material | |
| perfume | 0.35 | 6.7 | Intarome | Fragrance 83707 |
| Citric acid | 1.28 | 24.4 | ADM, H&R, | Granular Citric Acid |
| | | | WEGO, | |
| | | | Coleman | |
| Sodium citrate | 0.64 | 12.2 | Tate & Lyle | Sodium Citrate Dihydrate |
| viscosity control | 0.2 | 3.8 | Kelco | Kelzan T |
| agent | | | | |
| Dye/colorant | 0.003 | 0.048 | Chromatech | Chromatint Violet X-0160 |

While specific examples have been described in the specification and illustrated in the drawings, it will be understood by those skilled in the art that various changes may be made and equivalence may be substituted for elements thereof without departing from the scope of the present teachings as defined in the claims. Furthermore, the mixing and matching of features, elements and/or functions between various examples may be expressly contemplated herein so that one skilled in the art would appreciate from the present teachings that features, elements and/or functions of one example may be incorporated into another example as appropriate, unless described otherwise above. Moreover, many modifications may be made to adapt a particular situation or material to the present teachings without departing from the essential scope thereof. Therefore, it may be intended that the present teachings not be limited to the particular examples illustrated by the drawings and described in the specification as the best mode of presently contemplated for carrying out the present teachings but that the scope of the present disclosure will include any embodiments following within the foregoing description and any appended claims.

What is claimed is:

1. A storage deodorant composition for deodorizing and storing a wastewater storage tank, the composition comprising:
(a) an odor control agent;
(b) a detergent;
(c) a surface active agent; and
(d) a lubricant,
wherein the storage deodorant composition is compatible with antifreeze agents.

2. The storage deodorant composition according to claim 1, further comprising one or more of a dispersion agent, a perfume and a dye.

3. The storage deodorant composition according to claim 1, wherein the amount of the odor control agent is about 0.1% to about 5.0 wt %, the amount of detergent is about 1.0 to about 5.0 wt %, the amount of surface active agent is about 0.1% to about 2.0 wt % and the amount of lubricant is about 0.05% to about 2.0 wt %.

4. A method for treating a wastewater storage tank during a period of non-use, the method comprising:
(a) adding a storage deodorant composition to a wastewater storage tank, the storage deodorant composition comprising an odor control agent, a detergent, a surface active agent, and a lubricant; and
(b) leaving the storage composition in the wastewater storage tank for the period of non-use.

5. The method according to claim 4, wherein leaving the storage composition in the wastewater storage tank includes leaving the storage composition in the wastewater storage tank for at least about one day.

6. The method according to claim 5, wherein leaving the storage composition in the wastewater storage tank includes leaving the storage composition in the wastewater storage tank for no longer than 12 months.

7. The method according to claim 4, wherein the composition is added to a wastewater storage tank selected from the group consisting of black water and gray water RV holding tanks, totes, marine and boat holding tanks, aircraft lavatory holding tanks and portable toilet holding.

8. The method according to claim 4, further comprising flushing an empty wastewater storage tank with a liquid prior to adding the storage deodorant composition.

9. The method according to claim 4, wherein adding the storage deodorant composition comprises adding the storage deodorant composition to a wastewater storage tank containing antifreeze solution.

10. A method for treating a wastewater storage tank during non-use, the method comprising:
(b) adding a solution comprising antifreeze to the wastewater tank;
(b) adding a storage deodorant composition to the wastewater storage tank, the storage deodorant composition comprising an odor control agent, a detergent, a surface active agent, and a lubricant; and
(c) leaving the storage deodorant composition in the wastewater storage tank for a period of non-use.

11. The method of claim 10, wherein the period of non-use is at least about one day.

12. The method of claim 10, wherein the period of non-use is between about one day and about 12 months.
